(19) United States
(12) Patent Application Publication
(10) Pub. No.: US 2013/0102071 A1
(43) Pub. Date: Apr. 25, 2013

(54) SYSTEM AND METHOD FOR PREPARING CELL CULTURE DISH MEDIA

(71) Applicants: Qiubao Pan, Singapore (SG); Yohanes Permana Rusli, Shanghai (CN)

(72) Inventors: Qiubao Pan, Singapore (SG); Yohanes Permana Rusli, Shanghai (CN)

(73) Assignee: TOKU-E Company, Bellingham, WA (US)

(21) Appl. No.: 13/335,751

(22) Filed: Dec. 22, 2011

(30) Foreign Application Priority Data
Oct. 21, 2011 (CN) PCT/CN2011/081132

(51) Int. Cl. C12M 1/22; B67D 7/78

(52) U.S. Cl. 435/305.1; 222/145.6; 222/1

(57) ABSTRACT

Systems for preparing cell culture dish media are provided which are well adapted for preparing cell culture media, supplements, additives and/or other ingredients in an efficient and versatile manner to form sterilized cell culture dish media having a variety of ingredients well adapted with respect to uniformity and nutritional content to culture microorganisms in a particularly effective manner. The systems include an array of filtration and steam sterilization devices to sterilize various types of media for selective combination of one or more of the types of media in a mixing chamber to form cell culture dishes (e.g., agar plates). Methods of preparing cell culture dish media and cell culture dishes are also provided.

### CROSS-REFERENCES TO RELATED APPLICATIONS

This application claims priority to PCT Application Serial No. PCT/CN2011/081132 filed Oct. 21, 2011, wherein this application is incorporated herein by reference in its entirety.

### BACKGROUND

1. Technical Field

This disclosure generally relates to systems and methods for preparing cell culture dishes to culture microorganisms, and more particularly, to systems and methods for preparing sterilized cell culture dish media to form the cell culture dishes.

2. Description of the Related Art

Cell culture dish preparation systems are known which can conveniently prepare, sterilize and discharge certain media into cell culture dishes in an automated manner. These systems, however, suffer from a number of deficiencies and drawbacks including, for example, the lack of versatility with respect to the types of media that may be prepared. In addition, the systems typically involve autoclave sterilization processes which can be quite time consuming and can damage nutrients in the cell culture media. As a result, the effectiveness of the resultant cell culture dish media to grow certain microorganisms may be diminished. Moreover, the time that is required to prepare a single type of cell culture media is less than desirable and this problem is compounded when multiple different types of media may be desired, such as, for example, when running experiments with different parameters.

### BRIEF SUMMARY

Embodiments described herein provide systems and methods for preparing cell culture dishes which are particularly well adapted for sterilizing cell culture media, supplements and additives in an efficient and versatile manner to form cell culture dish media having a variety of compositions. In addition, the resultant sterilized cell culture dish media is well adapted with respect to uniformity and nutritional content to culture microorganisms in a particularly effective manner.

According to one embodiment, a system for preparing cell culture dish media may be summarized as including a primary filtration device having at least one membrane filter and a heat source, the filtration device being configured to receive an amount of a cell culture media and sterilize the cell culture media via a heated filtration process; a dispenser configured to selectively dispense an amount of a desired ingredient for mixing with the cell culture media; an intermediate receptacle coupled to the primary filtration device and the dispenser to selectively receive the cell culture media and the desired ingredient from the primary filtration device and the dispenser, respectively; and a mixing chamber positioned downstream of the intermediate receptacle to receive the cell culture media and the desired ingredient and mix the same during a mixing operation to form a cell culture dish media, the mixing chamber including an outlet to selectively discharge the cell culture dish media.

The system may further include a second filtration device positioned between the dispenser and the intermediate receptacle to selectively receive the desired ingredient from the dispenser and filter sterilize the desired ingredient prior to introduction into the intermediate receptacle. The second filtration device may be a supplement filtration device which is configured to receive and filter sterilize a supplement for mixing with the cell culture media or the second filtration device may be an additive filtration device which is configured to receive and filter sterilize a temperature sensitive additive at or below an ambient temperature for mixing with the cell culture media. The system may further include a third filtration device coupled to the intermediate receptacle, the third filtration device configured to receive an amount of another desired ingredient, sterilize the other desired ingredient via a filtration process and discharge the other desired ingredient into the intermediate receptacle. The primary filtration device, the second filtration device and the third filtration device may be arranged in a parallel relationship to discharge different media into the intermediate receptacle independently of each other.

The system may further include a second dispenser coupled to the intermediate receptacle to receive a non-filterable media and selectively dispense the non-filterable media into the intermediate receptacle for sterilization via pressurized steam. The system may further include a pressurized steam source coupled to the intermediate receptacle to selectively sterilize the intermediate receptacle with pressurized steam during a sterilization operation.

The system may further include a second heat source coupled to the intermediate receptacle to selectively maintain the cell culture media at an elevated temperature of at least 45° C. when the cell culture media is received in the intermediate receptacle.

The system may further include a mixing apparatus upstream of the primary filtration device to prepare the cell culture media for introduction into the primary filtration device, the mixing apparatus including a cell culture media powder dispenser and a distilled water dispenser for selectively dispensing amounts of cell culture media powder and distilled water into a mixing chamber of the mixing apparatus. The mixing apparatus may include a heating element configured to maintain the cell culture media at an elevated temperature of at least 45° C. while discharging the cell culture media from the mixing chamber to the primary filtration device.

The system may further include a pressurized air source coupled to at least the primary filtration device to selectively urge cell culture media through the at least one membrane filter during a filtration operation. The pressurized air source may be configured to subject the cell culture media at least temporarily to a pressure between about 0.1 MPa and about 0.7 MPa to urge the cell culture media through the at least one membrane filter.

The primary filtration device may include a plurality of membrane filters of different porosities including an upstream membrane filter to filter particles of a size greater than a first threshold size and a downstream membrane filter to filter particles of a size greater than a second threshold size, the first threshold size of the upstream membrane filter being between about 0.35 μm and about 1.20 μm and the second threshold size of the downstream membrane filter being between about 0.10 μm and about 0.40 μm.

The system may further include a plurality of valves, at least one valve coupled between the intermediate receptacle and each of the primary filtration device and the mixing chamber; and a control system coupled to the plurality of valves to selectively control the amount of the cell culture media introduced into the intermediate receptacle and discharged into the mixing chamber. The control system may be further coupled to an air release valve to selectively release pressurized air into the primary filtration device to urge cell culture media through the at least one membrane filter during a filtration operation. The control system may be further coupled to a steam release valve to selectively release pressurized steam into the intermediate receptacle. The control system may be further coupled to the heat source of the primary filtration device to regulate a temperature of the cell culture media as the cell culture media passes through the at least one membrane filter during a filtration operation. The heat source may be configured to maintain the temperature of the cell culture media at or above about 45° C. as the cell culture media passes through the at least one membrane filter during the filtration operation. The control system may be further coupled to a discharge valve at the outlet of the mixing chamber to selectively discharge the cell culture dish media. The control system may be further coupled to a conveyance system to selectively position a series of cell culture dishes intermittingly below the outlet of the mixing chamber to selectively receive the cell culture dish media to form cell culture dishes.

A method of preparing cell culture dish media may be summarized as including sterilizing cell culture media through at least one membrane filter of a primary filtration device in a heated filtration process; discharging the sterilized cell culture media into a mixing chamber downstream of the primary filtration device; mixing the sterilized cell culture media with at least a first ingredient in the mixing chamber to form a cell culture dish media; and selectively discharging the cell culture dish media from the mixing chamber to form one or more cell culture dishes.

The method may further include sterilizing the first ingredient through a second filtration device prior to introduction in the mixing chamber. The second filtration device may be, for example, a supplement filtration device for filter sterilizing supplements or an additive filtration device for sterilizing a temperature sensitive additive at or below an ambient temperature.

The method may further include mixing the sterilized cell culture media with at least a second ingredient in the mixing chamber to form a cell culture dish media; and sterilizing the second ingredient through a third filtration device prior to introduction in the mixing chamber.

The method may further include discharging the sterilized cell culture media into an intermediate receptacle prior to introduction into the mixing chamber; and sterilizing the intermediate receptacle with pressurized steam after the sterilized cell culture media is discharged into the mixing chamber.

The method may further include dispensing a non-filterable cell culture media into an intermediate receptacle; and introducing pressurized steam into the intermediate receptacle to sterilize the non-filterable cell culture media.

The method may further include maintaining the cell culture media at an elevated temperature of at least about 45° C. while discharging the cell culture media into the primary filtration device. The method may further include maintaining the cell culture media at an elevated temperature of at least about 45° C. while sterilizing the cell culture media through the at least one membrane filter of the primary filtration device. The method may further include maintaining the sterilized cell culture media at an elevated temperature of at least about 45° C. while the cell culture media is received in an intermediate receptacle prior to introduction into the mixing chamber. The method may further include maintaining the sterilized cell culture media at an elevated temperature of at least about 45° C. while the cell culture media is mixed in the mixing chamber.

The method may further include dispensing amounts of cell culture media powder and distilled water into a mixing chamber of an upstream mixing apparatus; and mixing the determined amounts of cell culture media powder and distilled water to prepare the cell culture media for introduction into the primary filtration device.

The method may further include urging the cell culture media through the at least one membrane filter of the primary filtration device with pressurized air.

The method may further include conveying a series of dishes intermittingly below an outlet of the mixing chamber to selectively receive the cell culture dish media to form cell culture dishes.

Sterilizing the cell culture media, discharging the sterilized cell culture media into the mixing chamber, mixing the sterilized cell culture media with at least the first ingredient in the mixing chamber to form a cell culture dish media and selectively discharging the cell culture dish media from the mixing chamber to form one or more cell culture dishes may be automated to produce one or more cell culture dishes each having a determined volume of the cell culture dish media in response to a preparation instruction.

### BRIEF DESCRIPTION OF THE SEVERAL VIEWS OF THE DRAWINGS

FIG. 1 is a schematic diagram of a system for preparing cell culture dish media, according to one embodiment.

FIG. 2 is a process flow diagram for preparing cell culture dish media in accordance with one embodiment.

FIG. 3 is a process flow diagram for preparing various types of cell culture dish media in accordance with one embodiment.

FIGS. 4A and 4B are a process flow diagram for preparing agar cell culture dish media in accordance with one embodiment.

FIGS. 5A and 5B are a process flow diagram for preparing a non-filterable cell culture dish media in accordance with one embodiment.

FIG. 6 is a process flow diagram for preparing a supplement for combination with cell culture media in accordance with one embodiment.

FIG. 7 is a process flow diagram for preparing a temperature sensitive additive for combination with cell culture media in accordance with one embodiment.

### DETAILED DESCRIPTION

In the following description, certain specific details are set forth in order to provide a thorough understanding of various disclosed embodiments. However, one skilled in the relevant art will recognize that embodiments may be practiced without one or more of these specific details. In other instances, well-known structures or steps associated with filtration and sterilization equipment and filtration and sterilization processes may not be shown or described in detail to avoid unnecessarily obscuring descriptions of the embodiments.

For instance, it will be appreciated by those of ordinary skill in the relevant art that the systems described herein can be configured to provide automated or semi-automated preparation of sterilized cell culture dish media by incorporating an appropriate control and monitoring system, including for example, a control and monitoring system having temperature and/or pressure sensors, flow control valves and other devices to monitor and control the operation of the system in an automatic or semi-automatic manner. The control and monitoring system may be controlled, for example, via a programmable logic controller (PLC) or other suitable controller to prepare the resultant cell culture dish media in a particularly efficient manner.

Unless the context requires otherwise, throughout the specification and claims which follow, the word “comprise” and variations thereof, such as, “comprises” and “comprising” are to be construed in an open, inclusive sense, that is as “including, but not limited to.”

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

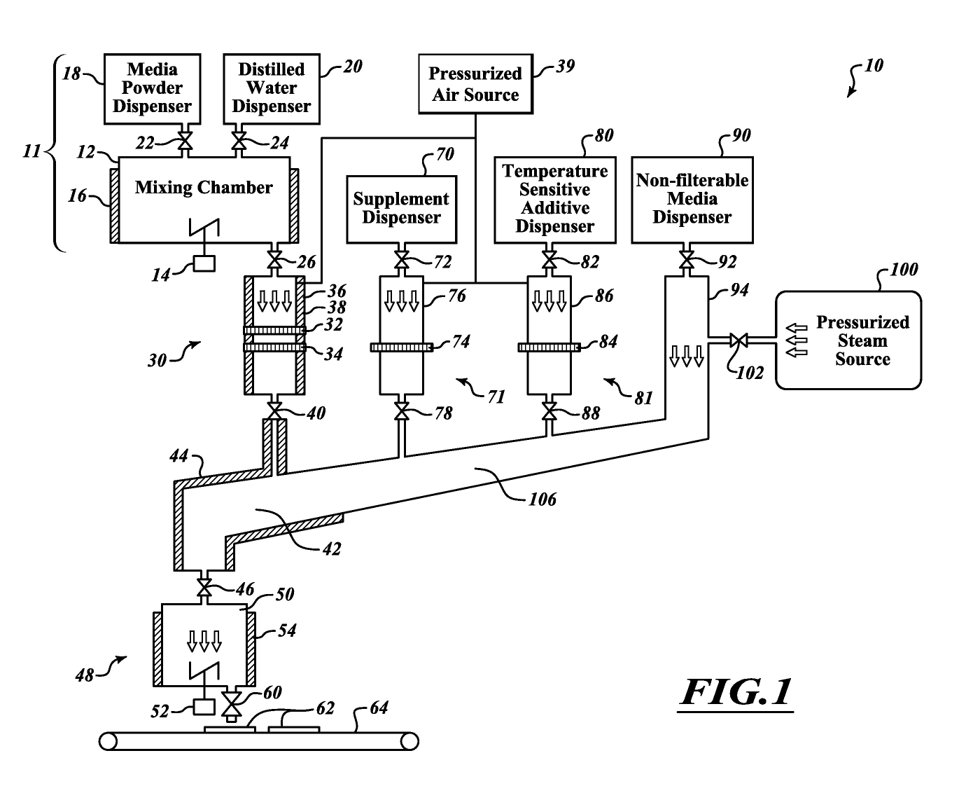

FIG. 1 is a schematic diagram of a cell culture dish media preparation system 10 according to one example embodiment. The preparation system 10 includes a mixing apparatus 11 for preparing agar cell culture media or broth media for subsequent sterilization and optional combination with one or more supplements and/or additives to form a cell culture dish media for culturing microorganisms in a cell culture dish (e.g., Petri dish) or similar device. The mixing apparatus 11 includes a mixing chamber 12 for combining dehydrated culture media with distilled water to form a cell culture media solution, such as, for example, an agar cell culture media solution or broth media solution. The mixing apparatus 11 includes a stirrer or mixing device 14 for this purpose. The mixing chamber 12 is also coupled to or otherwise includes a heat source 16 for selectively heating and/or maintaining the contents within the mixing chamber 12 at an elevated temperature, if desired, prior to discharging the cell culture media from the mixing apparatus 11. For example, in some embodiments, when agar cell culture media is prepared, the heat source 16 is configured to maintain the temperature of the agar cell culture media at or above 45° C. as the agar cell culture media is discharged from the mixing apparatus 11. In other instances, the heat source 16 may remain idle when preparing certain media, such as, for example, broth media. Accordingly, the contents of the mixing apparatus may be mixed and discharged at or near an ambient temperature in some embodiments.

The mixing apparatus 11 further includes a cell culture media powder dispenser 18 and a distilled water dispenser 20 for selectively dispensing determined amounts of cell culture media powder and distilled water, respectively, into the mixing chamber 12 of the mixing apparatus 11. Each of the cell culture media powder dispenser 18 and the distilled water dispenser 20 may be coupled to the mixing chamber 12 with a respective volume regulator 22, 24 or other control valve to selectively control the amount of cell culture media powder and distilled water dispensed into the mixing chamber 12. In addition, the volume regulators 22, 24 or other control valves of the cell culture media powder dispenser 18 and distilled water dispenser 20 may be communicatively coupled to a control system for automated control of the control valves 22, 24 in response to a command to prepare, for example, a desired volume of cell culture media having a desired consistency and/or level of saturation. A discharge valve 26 is positioned in the mixing chamber 12 to selectively discharge determined amounts of the prepared cell culture media. The discharge valve 26 may likewise be communicatively coupled to the control system for automated control and dispensing of determined volumes of the prepared cell culture media.

Although the illustrated embodiment of the cell culture dish media preparation system 10 includes a mixing apparatus 11 for preparing cell culture media from a dehydrated powder form, it is appreciated that in other embodiments, the cell culture media solution may be prepared remotely from the preparation system 10, such as, for example, in a separate mixing device. It is beneficial, however, to provide an integrated system in which the mixing apparatus 11 is coupled to other components of the preparation system 10 so as to minimize exposure of the cell culture media to contaminants and reduce material handling demands, among other reasons.

The preparation system 10 further includes a primary filtration device 30 for sterilizing the cell culture media via a filtration process, and in some embodiments, a heated, multi-stage filtration process. The primary filtration device 30 is positioned downstream of the mixing apparatus 11 to receive the prepared cell culture media. In some embodiments, the primary filtration device 30 includes a plurality of membrane filters 32, 34 of different porosities supported in a filtration housing 36 to filter sterilize the cell culture media in a multi-stage filtration process. In other embodiments, the primary filtration device 30 may include a single membrane filter supported in the filtration housing 36 to filter sterilize the cell culture media in a single stage filtration process. The primary filtration device 30 further includes a heat source 38 to selectively heat and/or maintain contents within the housing 36 at an elevated temperature during at least a portion of the filtration operation. For instance, in some embodiments, the heat source is configured to maintain an agar cell culture media in the filtration device at or above a gelling point or a melting point of the agar cell culture media. In some embodiments, for example, the heat source may be configured to maintain an agar cell culture media in the filtration device at or above 45° C. as the agar cell culture media is filter sterilized. To aid in the filtration operation, a pressurized air source 39 may be coupled to the filtration housing 36 to selectively pressurize an interior chamber of the housing 36 and urge contents within the interior chamber through the series of membrane filters 32, 34. A discharge valve 40 is coupled to the filtration housing 36 to selectively discharge the sterilized cell culture media into an intermediate chamber or receptacle 42 of the preparation system 10.

According to some embodiments, the primary filtration device 30 and other filtration devices described herein may include features or aspects of the filtration devices and methods described in Applicants' co-pending application filed concurrently on Oct. 21, 2011 and entitled “Agar Based Growth Medium Filtration Device and Method” and assigned Seed IP Docket No. 900197.402PC, which application is incorporated herein by reference in its entirety. For example, in some embodiments of the present invention, the primary filtration device 30 may include two sequential membrane filters 32, 34 of different porosities positioned within the filtration housing 36 in which an upstream one of the membrane filters 32 has a porosity to filter particles of a size greater than a first threshold size and a downstream one of the membrane filters 34 has a porosity to filter particles of a size greater than a second threshold size that is less than the first threshold size. In some embodiments, the first threshold size of the upstream membrane filter 32 may be between about 0.35 μm and about 1.20 μm and the second threshold size of the downstream membrane filter 34 may be between about 0.10 μm and about 0.40 μm but at least 0.20 μm less than the first threshold size of the upstream membrane filter 32. In some embodiments, the first threshold size of the upstream membrane filter 32 may be about 0.45 μm±0.03 μm and the second threshold size of the downstream membrane filter 34 may be about 0.22 μm±0.03 μm.

As another example, the primary filtration device 30 may be configured to filter agar cell culture media in a heated, multi-stage process in which the agar cell culture media is maintained at or above a gelling point or melting point of the agar cell culture media. In some embodiments, for example, the primary filtration device 30 may be configured to filter agar cell culture media in a heated, multi-stage process in which the agar cell culture media is maintained at or above 45° C. during the filtration operation and subjected to a pressure of between about 0.1 MPa and about 0.7 MPa to urge the agar cell culture media through a series of membrane filters 32, 34 offset from each other, each membrane filter being configured to filter particles of a different particle size. In other embodiments, the agar cell culture media may be maintained between about 60° C. and 100° C. as the cell culture media is forced to pass sequentially through the filter units 32, 34. In still other embodiments, the agar cell culture media may be maintained between about 85° C. and 100° C. as the cell culture media is forced to pass sequentially through the membrane filters 32, 34. In some embodiments, the agar cell culture media may be subjected to a pressure of between about 0.40 MPa±0.05 MPa to urge the agar cell culture media through the series of membrane filters 32, 34.

A portion of the intermediate chamber or receptacle 42 may be coupled to another heat source 44 such that at least that portion of the chamber or receptacle 42 may be selectively heated and/or maintained at an elevated temperature while containing certain cell culture media, such as, for example, agar cell culture media. The intermediate chamber of receptacle 42 may slope downwardly toward a discharge valve 46 at a terminal end of the intermediate chamber of the receptacle 42. In this manner, the sterilized cell culture media may be fed toward the discharge valve 46 of the intermediate chamber or receptacle 42 at least partially under the influence of gravity. Other transport mechanisms including, for example, a pressurized air source providing a positive pressure on the contents of the intermediate chamber or receptacle 42 or a vacuum source generating a vacuum to draw contents through the intermediate chamber or receptacle 42 may be provided to assist in moving the sterilized cell culture media through the intermediate chamber or receptacle 42.

From the intermediate chamber or receptacle 42, the sterilized cell culture media is discharged into another mixing apparatus 48 for optional combination with one or more supplements and/or additives prior to discharging the sterilized cell culture media from the preparation system 10. The mixing apparatus 48 includes a mixing chamber 50 to receive the sterilized cell culture media and the optional supplements and/or additives. In addition, the mixing apparatus 48 includes a stirrer or mixing device 52 to selectively mix the contents to form a generally uniform or consistent product. A heat source 54 may also be coupled to the mixing chamber 50 to enable mixing of the contents under a heated condition. More particularly, the heat source 54 may be configured to supply sufficient heat to maintain the contents thereof in a generally flowable condition. The heat source 54, for example, is advantageous when processing certain types of cell culture media, such as, for example, agar cell culture media. A discharge valve 60 is positioned in the mixing chamber 50 to selectively discharge the sterilized cell culture media. The discharge valve 60 may be communicatively coupled to the control system for control and dispensing of determined volumes of the sterilized cell culture media in an automated or semi-automated manner.

In some embodiments, the sterilized cell media may be dispensed into a cell culture dish 62, such as, for example, a Petri dish, to form a cell culture dish or plate (e.g., an agar plate). In some embodiments, a conveyance system 64 may be provided to selectively position a series of cell culture dishes 62 intermittingly below the outlet of the discharge valve 60 to selectively receive the cell culture dish media in an automated or semi-automated manner.

As shown in FIG. 1, the example embodiment of the cell culture dish media preparation system 10 further includes a supplement dispenser 70. The supplement dispenser 70 is coupled to the intermediate chamber or receptacle 42 with a supplement filtration device 71 in parallel with the primary filtration device 30. The supplement dispenser 70 is configured to hold a supply of a supplement and selectively discharge the supplement into the supplement filtration device 71 through a discharge valve 72. The discharge valve 72 may be communicatively coupled to the control system for automated control and dispensing of determined volumes of the supplement into the supplement filtration device 71. The supplement filtration device 71 includes at least one membrane filter 74 supported within a filtration housing 76. The membrane filter(s) 74 are configured to sterilize the supplement via a filter sterilization process by filtering particles greater than a threshold size. For example, in some embodiments, a single membrane filter 74 is provided which is configured to filter particles of a size greater than a threshold size of between about 0.10 μm and about 0.25 μm. The pressurized air source 39 or another pressurized air source may be coupled to the filtration housing 76 of the supplemental filter device 71 to assist in passing the supplement through the membrane filter(s) 74.

Upon filtration, the supplement may be temporarily retained within a downstream portion of the supplement filtration device 71 prior to discharging the supplement into the intermediate chamber or receptacle 42. Ultimately, the sterilized supplement may be discharged through a discharge valve 78 coupled to the filtration housing 76 into the intermediate chamber or receptacle 42 of the preparation system 10 and then into the mixing apparatus 48 via discharge valve 46. In this manner, supplements, such as, for example, vitamins (e.g., vitamin A, B6, B12, E, etc.) may be sterilized by the supplement filtration device 71 and selectively added as desired to a volume of sterilized cell culture media and mixed appropriately to provide a sterilized cell culture media of a desired composition. The mixture may be dispensed into a cell culture dish, such as, for example, a Petri dish, to form a cell culture dish or plate (e.g., an agar plate). In some embodiments, the mixture may be prepared and dispensed into one or more cell culture dishes in a semi-automated or fully automated manner. For instance, in response to an instruction to prepare a desired composition of a cell culture media, the control system may activate the various valves 22, 24, 26, 40, 46, 60, 72, 78, the mixing devices 14, 52, pressurized air source 39 and other components (e.g., heat sources 16, 38, 44, 54) in a coordinated manner to dispense a determined volume of sterilized cell culture media and a determined volume of one or more supplements into the mixing apparatus 48 and mix and dispense the same without further user interaction.

In other embodiments, the cell culture dish media preparation system 10 may include a supplement dispenser 70 which is coupled to the intermediate chamber or receptacle 42 without a supplement filtration device 71 positioned therebetween. Instead, the supplement dispenser 70 may be configured to receive a pre-sterilized supplement and dispense the supplement into the intermediate chamber or receptacle 42 and/or the mixing apparatus 48 without filter sterilization. For example, in some embodiments, the discharge valve 72 of the supplement dispenser 70 may be coupled directly to the intermediate chamber or receptacle 42 or to the mixing apparatus 48.

As shown in FIG. 1, the example embodiment of the cell culture dish media preparation system 10 further includes a temperature sensitive additive dispenser 80. Temperature sensitive additives include cell culture media additives which may, for example, degrade upon exposure to elevated temperatures, and include, for example, antibiotics (e.g., penicillin, streptomycin, gentamicin, etc.). The additive dispenser 80 is coupled to the intermediate chamber or receptacle 42 with an additive filtration device 81 in parallel with the primary filtration device 30 and the supplement filtration device 71. The additive dispenser 80 is configured to hold a supply of a temperature sensitive additive and selectively discharge the additive into the additive filtration device 81 through a discharge valve 82. The discharge valve 82 may be communicatively coupled to the control system for automated control and dispensing of determined volumes of the additive into the additive filtration device 81. The additive filtration device 81 includes at least one membrane filter 84 supported within a filtration housing 86. The membrane filter(s) 84 are configured sterilize the additive via a filter sterilization process by filtering particles greater than a threshold size. For example, in some embodiments, a single membrane filter 84 is provided which is configured to filter particles of a size greater than a threshold size of between about 0.10 μm and about 0.25 μm. The pressurized air source 39 or another pressurized air source may be coupled to the filtration housing 86 of the additive filtration device 81 to assist in passing the additive through the membrane filter(s) 84 to filter sterilize the additive.

Upon filtration, the additive may be temporarily retained within a downstream portion of the additive filtration device 81 prior to discharging the additive into the intermediate chamber or receptacle 42. Ultimately, the sterilized additive may be discharged through a discharge valve 88 coupled to the filtration housing 86 into the intermediate chamber or receptacle 42 of the preparation system 10 and then into the mixing apparatus 48 via discharge valve 46. In this manner, additives, such as, for example, an antibiotic (e.g., penicillin, streptomycin, gentamicin, etc.) may be sterilized by the additive filtration device 81 and selectively combined as desired to a volume of sterilized cell culture media and mixed appropriately to provide a sterilized cell culture media of a desired composition. The mixture may be dispensed into a cell culture dish, such as, for example, a Petri dish, to form a cell culture dish or plate (e.g., an agar plate). In some embodiments, the mixture may be prepared and dispensed into one or more cell culture dishes in a semi-automated or fully automated manner. For instance, in response to an instruction to prepare a desired composition of a cell culture dish media, the control system may activate the various valves 22, 24, 26, 40, 46, 60, 72, 78, 82, 88, the mixing devices 14, 52, pressurized air source 39 and other components (e.g., heat sources 16, 38, 44, 54) in a coordinated manner to dispense a determined volume of sterilized cell culture media and a determined volume of one or more desired supplements and/or one or more desired additives into the mixing apparatus 48 and mix and dispense the same without further user interaction.

In other embodiments, the cell culture dish media preparation system 10 may include a temperature sensitive additive dispenser 80 which is coupled to the intermediate chamber or receptacle 42 without an additive filtration device 81 positioned therebetween. Instead, the temperature sensitive additive dispenser 80 may be configured to receive a pre-sterilized additive and dispense the additive into the intermediate chamber or receptacle 42 and/or the mixing apparatus 48 without filter sterilization. For example, in some embodiments, the discharge valve 82 of the additive dispenser 80 may be coupled directly to the intermediate chamber or receptacle 42 or to the mixing apparatus 48.

As shown in FIG. 1, the example embodiment of the cell culture dish media preparation system 10 may further include a media dispenser 90 for non-filterable media, such as, for example, soybean milk, starch or corn meal media. The media dispenser 90 is coupled to the intermediate chamber or receptacle 42 via a column 94 and is configured to discharge the non-filterable media into column 94 through a discharge valve 92. The discharge valve 92 may be communicatively coupled to the control system for automated control and dispensing of determined volumes of the non-filterable media into the column 94. A pressurized steam source 100 is coupled to the column 94 via a valve 102 for selectively introducing pressurized steam into the column 94 during a sterilization operation. In some embodiments, the column 94 may be aligned generally in parallel with the primary filtration device 30, supplement filtration device 71 and additive filtration device 81.

The column 94 may be an upstream portion of the intermediate chamber or receptacle 42. The pressurized steam from the pressurized steam source 100 is used to sterilize the non-filterable media within the column 94 and/or within the intermediate chamber or receptacle 42 and assist in urging the non-filterable media toward the discharge valve 46 and hence mixing apparatus 48. The pressurized steam may also be introduced into the intermediate chamber or receptacle 42 intermittingly to sterilize an internal cavity 106 of the intermediate chamber or receptacle 42. For instance, in some embodiments, pressurized steam may be introduced into the intermediate chamber or receptacle 42 prior to dispensing media from the primary filtration device 30, supplement filtration device 71, additive filtration device 81 or non-filterable media dispenser 90. In this manner, the intermediate chamber or receptacle 42 may be selectively sterilized, for example, between each of a series of media dispensing operations to protect against cross-contamination and the like.

Upon sterilization of the non-filterable media, the media may be temporarily retained within the intermediate chamber or receptacle 42 of the preparation system 10 and then discharged into the mixing apparatus 48 via discharge valve 46. In this manner, non-filterable media, such as, for example, soybean milk, starch or corn meal media, may be sterilized by the pressurized steam source 100 and selectively combined with one or more supplements and additives in the mixing apparatus 48 to form a sterilized cell culture media of a desired composition. The mixture may be dispensed into a cell culture dish, such as, for example, a Petri dish, to form a cell culture dish or plate. In some embodiments, the mixture may be prepared and dispensed into one or more cell culture dishes in a semi-automated or fully automated manner. For instance, in response to an instruction to prepare a desired composition of a cell culture dish media, the control system may activate the various valves 46, 60, 72, 78, 82, 88, 92, 102, the mixing devices 14, 52, pressurized air source 39, the pressurized steam source 100 and other components in a coordinated manner to dispense a determined volume of sterilized non-filterable cell culture media, a determined volume of one or more desired supplements and/or one or more desired additives, and mix and dispense the same without further user interaction.

Although the illustrated embodiment shows three separate filtration devices 30, 71, 81 for different classes of media, it is appreciated that more or fewer filtration devices may be provided depending on the desired versatility of the system 10. In addition, although the illustrated embodiment includes a dispenser for non-filterable cell culture media, it is appreciated that in some embodiments, the non-filterable media dispenser may be omitted. Still further, although the illustrated embodiment includes filtration devices 71, 81 to filter sterilize various supplements and additives for admixture with the cell culture media of the primary filtration device 30, it is appreciated as discussed above that the supplement and additive dispensers 70, 80 may be coupled to the intermediate chamber or receptacle 42 or the mixing apparatus 48 without an intermediate filtration device 71, 81. The illustrated embodiment, however, provides a particularly versatile system 10 that is configured to enable the combination of numerous commercially available ingredients that are typically used to form cell culture dishes, including, for example, agar cell culture media, vitamins and various antibiotics. Furthermore, the system 10 enables a user to quickly and efficiently switch or adjust the composition of the prepared cell culture media without requiring time intensive autoclave sterilization and gamma sterilization processes.

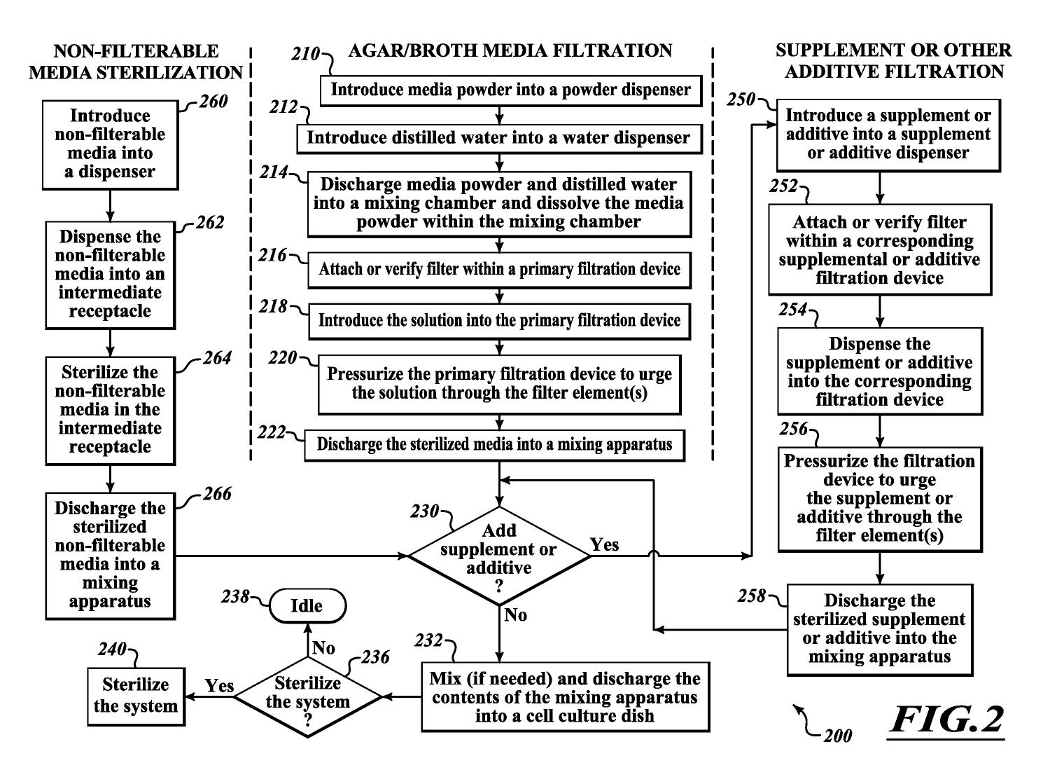

FIG. 2 shows a process flow diagram for preparing cell culture dish media according to one example embodiment. The process 200 may begin at 210 where cell culture media powder is introduced into a powder dispenser. At 212, distilled water may be introduced into a water dispenser. In some embodiments, however, the distilled water may be introduced into the water dispenser prior to or simultaneously with introduction of the media powder into the powder dispenser. The process continues at 214 where the media powder and water is discharged into a mixing chamber and mixed to dissolve the media powder and form a cell culture media solution. The process may continue at 216 by attaching one or more filter elements within a primary filtration device or otherwise verifying that the one or more filter elements is/are present. Next, at 218, the cell culture media solution is introduced into the primary filtration device from the mixing chamber. At 220, the primary filtration device is pressurized to urge the solution through the one or more filter elements to sterilize the solution. At 222, the sterilized cell culture media solution is discharged into a downstream mixing apparatus.

At decision box 230 it is determined whether a supplement or additive is to be combined with the sterilized cell culture media solution within the mixing apparatus. If no supplement or additive is to be added, then the sterilized cell culture media solution may be discharged at 232 from the mixing apparatus into one or more cell culture dishes without initiating a mixing operation. At decision box 236 it is then determined whether to sterilize the filtration system. If not, the system remains idle at 238. If so, a system sterilization process is initiated at 240 and pressurized steam is introduced into the system at an elevated temperature for an extended duration. For example, in some embodiments, pressurized steam at or above about 115° C. may be introduced into the system for about three to five minutes to sterilize the system effectively. After the sterilization process is complete, the steam may be vented.

If one or more supplements or additives are to be introduced, the process continues at 250 where one or more supplements or additives are introduced into a corresponding dispenser. The process may continue at 252 by attaching one or more filter elements within a supplemental or additive filtration device or otherwise verifying that the one or more filter elements is/are present. Next, at 254, a supplement or additive is dispensed into the corresponding filtration device. At 256, the supplement filtration device or additive filtration device is pressurized to urge the supplement or additive through the corresponding filtration device. The sterilized supplement or additive is then discharged into the mixing apparatus for combining with the sterilized cell culture media solution at 258. Alternatively, in some embodiments, pre-sterilized supplements and/or additives may be discharged into the mixing apparatus at 258 for combining with the sterilized cell culture media solution without the filtration steps 252, 254, 256. If additional supplements or additives are desired the process may return to 250. If no additional supplements or additives are desired, the contents of the mixing apparatus are then mixed and the resulting mixture is discharged into one or more cell culture dishes at 232. An optional sterilization process may then be performed at 236.

According to other embodiments, the process 200 may begin at 260 where a non-filterable media, such as, for example, soybean milk, starch or corn meal media, is introduced into a media dispenser. At 262, the non-filterable media may be dispensed or discharged into an intermediate chamber or receptacle for temporary storage and sterilization. At 264, the non-filterable media may be sterilized, such as, for example, introducing pressurized steam into the intermediate chamber or receptacle and exposing the non-filterable media to the pressurized steam for a determined duration at an elevated temperature, such as, for example, exposing the non-filterable media to pressurized steam at or above about 115° C. for about three to five minutes. Next, at 266, the sterilized non-filterable media may be discharged into a downstream mixing apparatus.

Similar to the discussion above, the process may then continue at decision box 230 to determine whether a supplement or additive is to be combined with the sterilized non-filterable media within the downstream mixing apparatus. If no supplement or additive is to be added, then the sterilized non-filterable media may be discharged at 232 from the mixing apparatus into one or more cell culture dishes without initiating a mixing operation. At decision box 236 it is then determined whether to sterilize the filtration system. If not, the system remains idle at 238. If so, a system sterilization process is initiated at 240 and pressurized steam is introduced into the system at an elevated temperature for an extended duration. For example, in some embodiments, pressurized steam at or above about 115° C. may be introduced into the system for about three to five minutes to sterilize the system effectively. After the sterilization process is complete, the steam may be vented.

If one or more supplements or additives are to be introduced, the process continues at 250 where one or more supplements or additives are introduced into a corresponding dispenser. The process may continue at 252 by attaching one or more filter elements within a supplemental or additive filtration device or otherwise verifying that the one or more filter elements is/are present. Next, at 254, a supplement or additive is dispensed into the corresponding filtration device. At 256, the supplement filtration device or additive filtration device is pressurized to urge the supplement or additive through the corresponding filtration device. The sterilized supplement or additive is then discharged into the mixing apparatus for combining with the sterilized non-filterable media at 258. Alternatively, in some embodiments, pre-sterilized supplements and/or additives may be discharged into the mixing apparatus at 258 for combining with the sterilized non-filterable media without the filtration steps 252, 254, 256. If additional supplements or additives are desired the process may return to 250. If no additional supplements or additives are desired, the contents of the mixing apparatus are then mixed and the resulting mixture is discharged into one or more cell culture dishes at 232. An optional sterilization process may then be performed at 236.

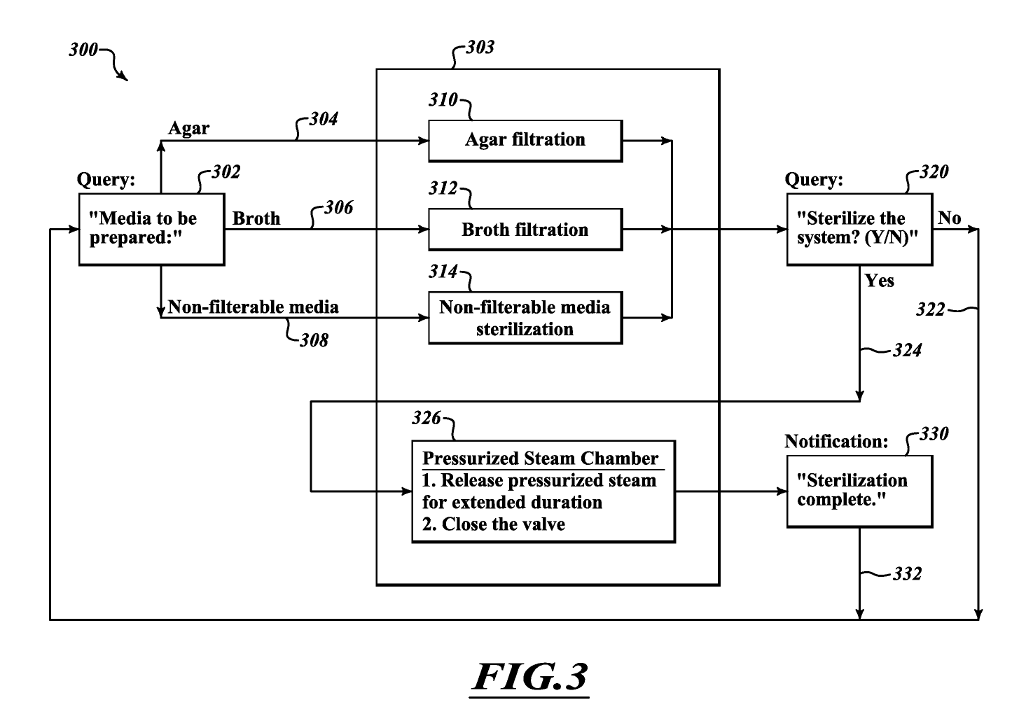

FIG. 3 shows a process flow diagram for preparing various types of cell culture dish media according to one example embodiment. The process 300 begins at 302 wherein the type of cell culture media to be prepared via a preparation system 303 is queried. If agar is selected as represented by process line 304, then an agar filtration method is performed at 310 that entails, among other things, introducing agar cell culture media into a cell culture dish media preparation system and sterilizing the agar cell culture media via a heated filtration process. If broth is selected as represented by process line 306, then a broth filtration method is performed at 312 that entails, among other things, introducing a broth media into the cell culture dish media preparation system and sterilizing the broth media via a filtration process. If a non-filterable media, such as, for example, a soybean milk, starch or corn meal media, is selected as represented by process line 308, then a non-filterable media sterilization method is performed at 314 that entails, among other things, introducing pressurized steam into the cell culture dish media preparation system and sterilizing the non-filterable media by subjecting it to the pressurized steam for an extended duration.

After the agar, broth or non-filterable media is prepared, the process continues at 320 with a query as to whether to sterilize the preparation system. If sterilization of the system is not desired as represented by process line 322, then the process 300 ends or may be repeated by returning to 302 and querying whether additional media of a certain type is to be prepared. If sterilization is desired, however, as represented by process line 324, then the process continues with a sterilization operation at 326. The sterilization operation may include introducing pressurized steam at or above about 115° C. into the system for about three to five minutes. In other embodiments, sterilization operation may include introducing pressurized steam into the system at lower temperatures for longer durations. After the sterilization operation is complete, the system may provide an indication or notification of the same at 330. The process 300 then ends or may be repeated by returning to 302 and querying whether additional media of a certain type is to be prepared.

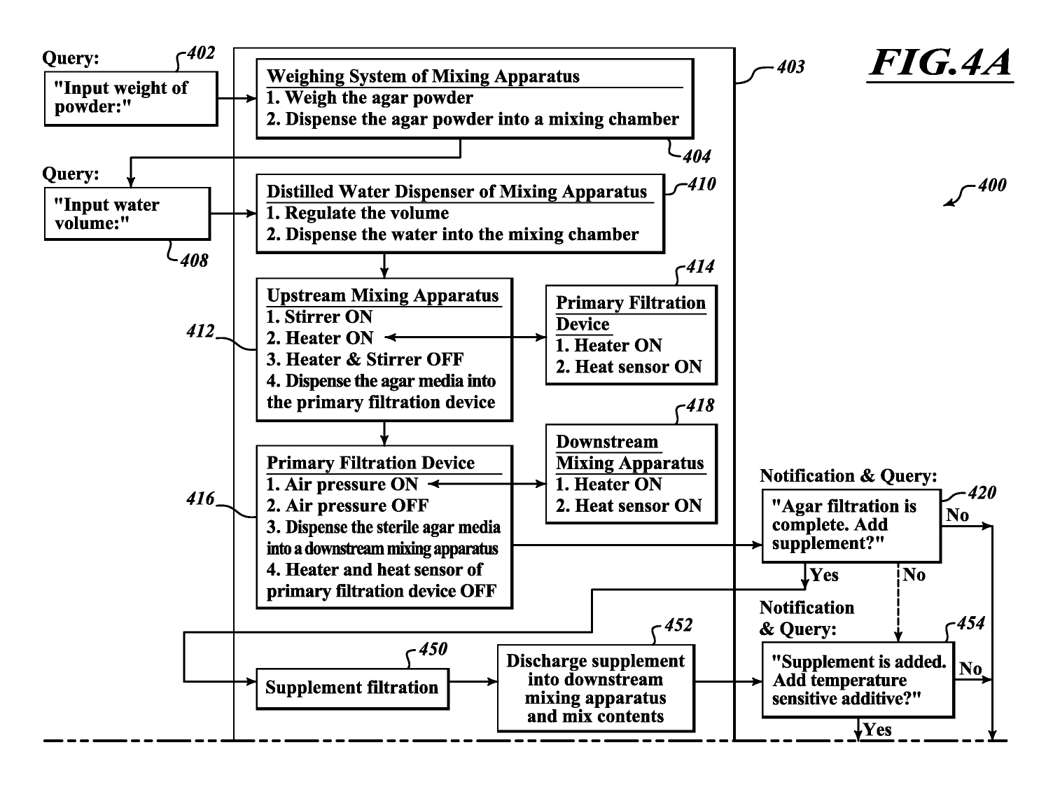

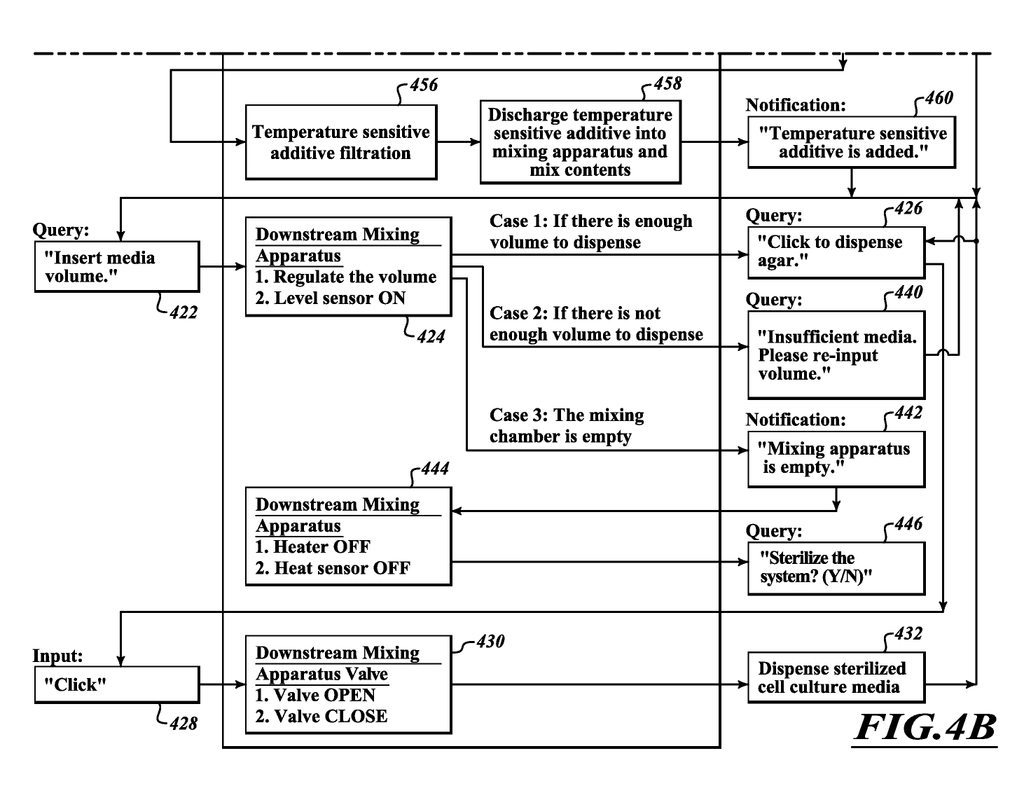

FIGS. 4A and 4B show a process flow diagram for preparing agar cell culture dish media according to one example embodiment. The process 400 begins at 402 where a query is generated to solicit the amount of agar powder to be processed by a preparation system 403. The query may seek input relating to the weight of the agar powder (as depicted in FIG. 4A), the volume of the agar powder or other information indicative of the amount of agar powder that is desired to be processed. Upon receiving such input, the corresponding amount of agar powder is apportioned from a supply of agar powder and discharged into a mixing apparatus for further processing. For instance, at 404 a weighing system may apportion an amount of agar powder corresponding to the desired amount indicated by the user and discharge it to a mixing apparatus of the system 403. At 408 a query is generated to solicit the volume of distilled water to be used in the process. The query may seek input relating to the weight of the water, the volume of the water (as depicted in FIG. 4A) or other information indicative of the amount of water that is required to be mixed with the agar powder. Upon receiving such input, the corresponding amount of water is apportioned from a supply of distilled water and discharged into the mixing apparatus with the agar powder for further processing. For instance, at 410 a regulator valve may be used to apportion an amount of distilled water corresponding to the desired amount indicated by the user and discharge it to the mixing apparatus. In other embodiments, the amount of water may be determined based on the amount of agar powder that is apportioned and a desired consistency or level of saturation that has been predetermined. For instance, in some embodiments, about 1000 ml of distilled water may be added for every 20 grams of agar powder. Accordingly, the user may not be required to specify the amount of distilled water to be dispensed into the system. In a similar manner, the system 403 may be configured to dispense predetermined amounts of agar powder and distilled water for producing a selected number of cell culture dishes. For instance, in some embodiments, a user may indicate that a particular quantity of dishes is desired and the corresponding volume of agar powder and distilled water to fulfill the request may be automatically dispensed into the mixing apparatus for additional processing.

At 412, a mixing device or stirrer of the mixing apparatus is activated to mix the contents therein. Concurrently, a heat source may be activated to raise and/or maintain the temperature of the contents at an elevated temperature that is sufficient to maintain the agar solution in a generally flowable state. In some embodiments, for example, the heat source is configured to maintain the agar solution in the mixing apparatus at or above 45° C. as the agar cell culture media is filter sterilized. In other embodiments, the agar cell culture media may be maintained between about 60° C. and 100° C. and in still other embodiments, the agar cell culture media may be maintained between about 85° C. and 100° C.

At 414, a heat source coupled to a primary filtration device may be activated to begin a pre-heating operation. One or more heat sensors may also be activated to monitor and control the temperature of the primary filtration device and any contents therein. The pre-heating operation may be synchronized with or otherwise coordinated with the heating operation performed in the mixing apparatus as depicted in FIG. 4A. In other embodiments, heating within the primary filtration device may be synchronized or otherwise coordinated with discharging the agar cell culture media solution into the primary filtration device or other actions. After the agar powder and distilled water is thoroughly mixed and heated to form a flowable agar cell culture media solution, the mixing device or stirrer and heat source of the mixing apparatus may be deactivated and the agar cell culture media solution may be discharged into the primary filtration device for subsequent sterilization.

At 416, pressurized air may be fed into the primary filtration device to urge the agar cell culture media solution through one or more membrane filters positioned within the primary filtration device. When more than one membrane filter is provided, the membrane filters may be offset from each other and positioned in the primary filtration device to filter progressively smaller particles with each successive membrane filter. For instance, in some embodiments, the primary filtration device may include two sequential membrane filters of different porosities positioned within a filtration housing in which an upstream one of the membrane filters has a porosity to filter particles of a size greater than a first threshold size and a downstream one of the membrane filters has a porosity to filter particles of a size greater than a second threshold size that is less than the first threshold size. In some embodiments, the first threshold size of the upstream membrane filter may be between about 0.35 μm and about 1.20 μm and the second threshold size of the downstream membrane filter may be between about 0.10 μm and about 0.40 μm but at least 0.20 μm less than the first threshold size of the upstream membrane filter. In some embodiments, the first threshold size of the upstream membrane filter may be about 0.45 μm±0.03 μm and the second threshold size of the downstream membrane filter may be about 0.22 μm±0.03 μm.

At 418, a heat source coupled to a mixing apparatus downstream of the primary filtration device may be activated to begin a pre-heating operation in the downstream mixing apparatus. One or more heat sensors may also be activated to monitor and control the temperature within the downstream mixing apparatus and any contents therein. The pre-heating operation may be synchronized with or otherwise coordinated with the pressurization operation performed in the primary filtration device as depicted in FIG. 4A. In other embodiments, heating within the downstream mixing apparatus may be synchronized or otherwise coordinated with discharging the sterilized agar cell culture media solution into the mixing apparatus or other actions. After the agar cell culture media solution is sterilized via the one or more membrane filters, the pressurized air source may be deactivated or otherwise isolated from the internal chamber of the primary filtration device. The heat source and one or more heat sensors of the primary filtration device may be deactivated and the agar cell culture media solution may be discharged from the primary filtration device toward and ultimately into the downstream mixing apparatus. At 420, an indication may be provided that the agar cell culture media preparation and sterilization process is complete. In addition, a query may be generated to request input indicative of whether a supplement is desired.

If no supplement is desired, the process continues at 422 where an additional query is generated to request input indicative of the amount of the agar cell culture media that is desired to be discharged. For instance, the query may seek input relating to the weight of the agar cell culture media, the volume of the agar cell culture media (as depicted in FIG. 4B) or other information indicative of the amount of agar cell culture media that is desired to be discharged. Upon receiving such input, an optional verification or level regulation process may be performed at 424 to ensure that there is sufficient supply of the sterilized cell culture media to fulfill the request. For instance, a level sensor may be provided to determine the amount of agar cell culture media available and compare the same with the amount of agar cell culture media requested to be discharged. In a first case, if the volume of available agar cell culture media meets or exceeds the requested amount, then the process may continue at 426 with prompting a user to confirm the request or “click” to discharge or dispense the sterilized agar cell culture media. At 428, the user confirms the request with a “click” of a button or other indication and the request is fulfilled at 430 where a discharge valve of the downstream mixing apparatus is cycled open and closed to dispense the agar cell culture media at 432. In other embodiments, the sterilized agar cell culture media may be discharged automatically without requiring further input from the user if there is sufficient supply. The process may end after the sterilized agar cell culture media is discharged or repeat by returning to 426 if sufficient supply remains in the mixing apparatus to fulfill a request for the same amount of sterilized agar cell culture media. Alternatively, the process may return to 422 to query the user whether a different amount of sterilized cell culture media may be desired.

If a supplement is desired, the process may continue from the supplement query at 420 with a supplement filtration process at 450, which involves filter sterilization of a supplement, such as, for example, one or more vitamins (e.g., vitamin A, B6, B12, E, etc.). At 452, the sterilized supplement is discharged into the downstream mixing apparatus and combined with the sterilized agar cell culture media in a mixing operation. Alternatively, in some embodiments, pre-sterilized supplements may be discharged into the downstream mixing apparatus and combined with the sterilized agar cell culture media without requiring the noted filtration process at 450. At 454, the process may continue with confirmation of the addition of the sterilized supplement and may be followed with a query to request input indicative of whether a temperature sensitive additive is desired. If no temperature sensitive additive is desired, the process continues to 422 where an additional query is generated to request input indicative of the amount of the agar cell culture media that is desired to be discharged, as described above. If on the other hand a temperature sensitive additive is desired, the process continues from the additive query at 454 with an additive filtration process at 456, which involves filter sterilization of a temperature sensitive additive, such as, for example, one or more antibiotics (e.g., penicillin, streptomycin, gentamicin, etc.). At 458 the sterilized additive is discharged into the downstream mixing apparatus and combined with the agar cell culture media and any supplements that may be present in a mixing operation. Alternatively, in some embodiments, pre-sterilized additives may be discharged into the downstream mixing apparatus and combined with the sterilized agar cell culture media without requiring the noted filtration process at 456. At 460, a confirmation may be provided indicating that the temperature sensitive additive has been added to the mixture. The process may then continue with a query for additional supplements and/or additives or may proceed to 422 where an additional query is generated to request input indicative of the amount of the agar cell culture media that is desired to be discharged, as described above. Once the desired amount of agar cell culture media is discharged, the process may end and the heat source and the one or more heat sensors of the downstream mixing apparatus may be deactivated and the entire system may be shut down.

According to the various embodiments of the process 400 described above, an agar based cell culture media may be prepared and sterilized with optional supplements and/or additives in a particularly efficient manner. It is appreciated that in other embodiments, broth type cell culture media may be prepared in a similar manner, but without the need in some embodiments for the heating operations described in connection with the upstream mixing apparatus, primary filtration device and downstream mixing apparatus which is otherwise provided to maintain the agar based cell culture media in a flowable state during the aforementioned process.

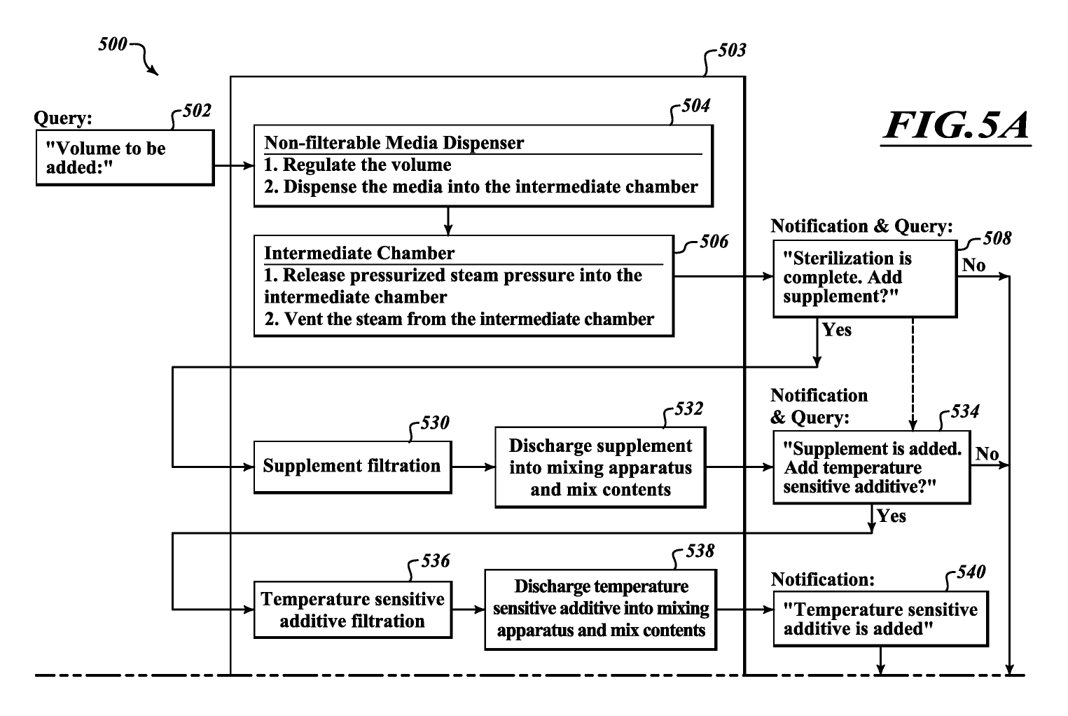

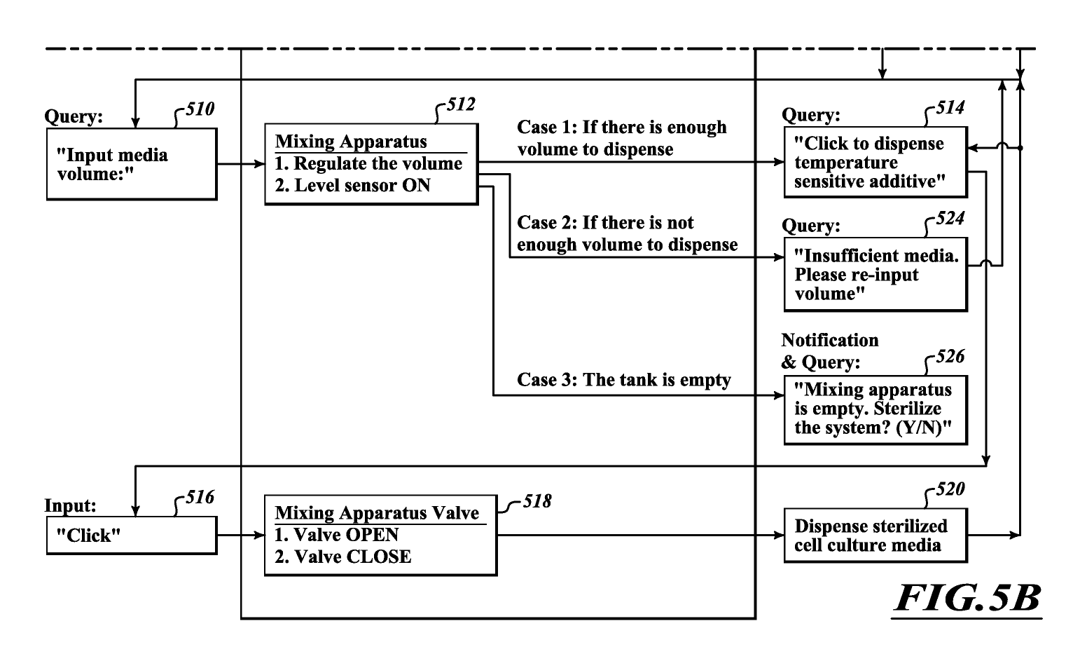

FIGS. 5A and 5B show a process flow diagram for preparing a non-filterable cell culture dish media according to one example embodiment. The process 500 begins at 502 where a query is generated to solicit the amount of the non-filterable cell culture media to be processed by a preparation system 503. The query may seek input relating to the weight of the of the non-filterable cell culture media, the volume of the of the non-filterable cell culture media (as illustrated in FIG. 5A) or other information indicative of the amount of non-filterable cell culture media that is desired to be processed. Upon receiving such input, the corresponding amount of non-filterable cell culture media is apportioned from a supply of non-filterable cell culture media at 504 and discharged into an intermediate chamber for further processing.

At 506, pressurized steam may be released into the intermediate chamber to subject the non-filterable cell culture media to the pressurized steam for a determined duration. For instance, in some embodiments, pressurized steam at or above about 115° C. may be introduced into the intermediate chamber for about three to five minutes to sterilize the non-filterable cell culture media. Upon sufficient exposure to sterilize the non-filterable cell culture media, the steam may be vented from the intermediate chamber, for example, via a steam release valve. At 508, an indication may be provided that the non-filterable cell culture media sterilization process is complete. In addition, a query may be generated to request input indicative of whether a supplement is desired.

If no supplement is desired, the process continues at 510 where an additional query is generated to request input indicative of the amount of the non-filterable cell culture media that is desired to be discharged. For instance, the query may seek input relating to the weight of the non-filterable cell culture media, the volume of the non-filterable cell culture media (as depicted in FIG. 5B) or other information indicative of the amount of non-filterable cell culture that is desired to be processed. Upon receiving such input, an optional verification or level regulation process may be performed at 512 to ensure that there is a sufficient supply of the sterilized non-filterable cell culture media to fulfill the request. For instance, a level sensor may be provided to determine the amount of the non-filterable cell culture media available and compare the same with the amount of the non-filterable cell culture media requested to be discharged.

In a first case, if the amount of available non-filterable cell culture media meets or exceeds the requested amount, then the process may continue at 514 with prompting a user to confirm the request or “click” to discharge or dispense the non-filterable cell culture media. At 516, the user confirms the request with a “click” of a button or other indication and the request is fulfilled at 518 in which a discharge valve of the mixing apparatus is cycled open and closed to dispense the non-filterable cell culture media at 520. In other embodiments, the non-filterable cell culture media may be discharged automatically without requiring further input from the user if there is sufficient supply. The process may end after the non-filterable cell culture media is discharged or repeat by returning to 514 if sufficient supply remains in the mixing apparatus to fulfill a request for the same amount of sterilized non-filterable cell culture media. Alternatively, the process may return to 510 to query the user whether a different amount of sterilized cell culture media may be desired. In a second case, if the amount of available non-filterable cell culture media is less than the requested amount, then the process may continue at 524 with prompting a user to input a request for a different volume as depicted in FIG. 5B or may prompt a user to add additional media by returning to the beginning of the process at 502. In a third case, if the mixing apparatus is empty, an indication may be provided at 526 to this effect and a user may be queried as to whether to sterilize the system. If so, the system may be sterilized with pressurized steam to prepare the system for storage or subsequent reuse.

If a supplement is desired, however, the process continues from the supplement query at 508 with a supplement filtration process at 530, which involves filter sterilization of a supplement, such as, for example, one or more vitamins (e.g., vitamin A, B6, B12, E, etc.). At 532, the sterilized supplement is discharged into the mixing apparatus and combined with the sterilized non-filterable cell culture media in a mixing operation. At 534, the process may continue with confirmation of the addition of the sterilized supplement and follow with a query to request input indicative of whether a temperature sensitive additive is desired. If no temperature sensitive additive is desired, the process continues to 510 where an additional query is generated to request input indicative of the amount of the non-filterable cell culture media that is desired to be discharged, as described above. If on the other hand a temperature sensitive additive is desired, the process continues from the additive query at 534 with a additive filtration process at 536, which involves filter sterilization of a temperature sensitive additive, such as, for example, one or more antibiotics (e.g., penicillin, streptomycin, gentamicin, etc.). At 538 the sterilized additive is discharged into the mixing apparatus and combined with the non-filterable cell culture media and any supplements that may be present in a mixing operation. At 540, a confirmation may be provided indicating that the temperature sensitive additive has been added to the mixture. The process may then continue with a query for additional supplements and/or additives or may proceed to 510 where an additional query is generated to request input indicative of the amount of the non-filterable cell culture media (inclusive of any supplements or additives that may be present) that is desired to be discharged, as described above. Once the desired amount of non-filterable cell culture media is discharged, the process may end and the mixing apparatus may be deactivated and the entire system may be shut down.

According to the various embodiments of the process 500 described above, non-filterable cell culture media may be prepared and sterilized with optional supplements and/or additives in a particularly efficient manner.

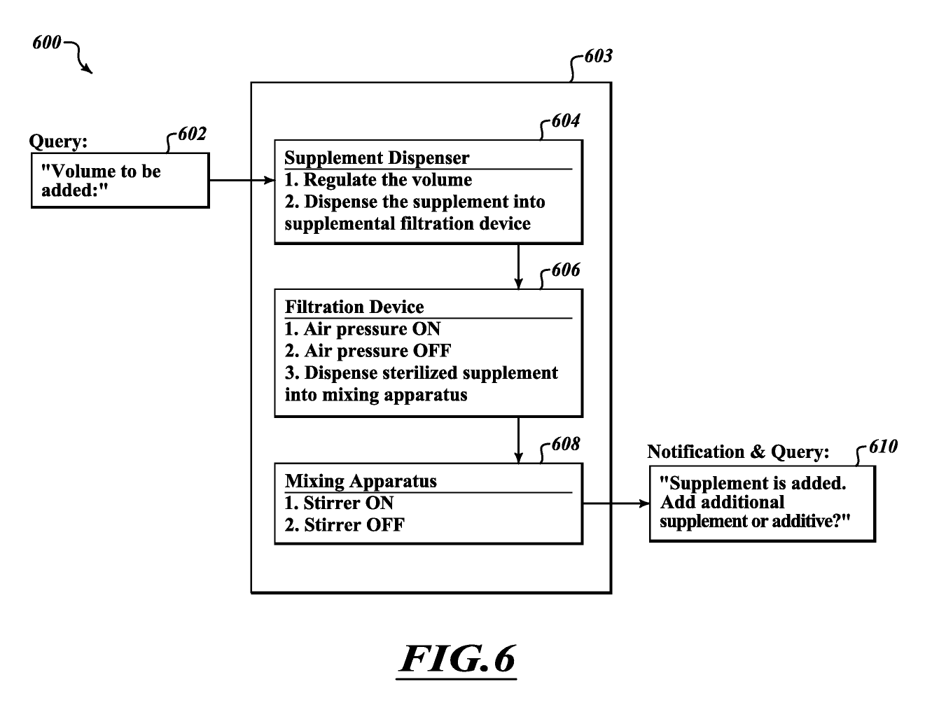

FIG. 6 shows a process flow diagram for preparing a supplement for combination with an agar cell culture media or other cell culture media according to one example embodiment. The process 600 begins at 602 where a query is generated to solicit the amount of the supplement to be processed by a preparation system 603. The query may seek input relating to the weight of the supplement, the volume of the supplement (as illustrated in FIG. 6) or other information indicative of the amount of the supplement that is desired to be processed. Upon receiving such input, the corresponding amount of the supplement is apportioned from a supply of the supplement at 604 and discharged into a supplement filtration device for further processing.

At 606, an internal chamber of the supplement filtration device is pressurized to urge the supplement through at least one filter element to sterilize the supplement. After the supplement is sterilized, the pressurized air source may be deactivated or otherwise isolated from the internal chamber of the supplement filtration device. The supplement may be discharged from the supplement filtration device toward and ultimately into the mixing apparatus. At 608, a stirrer or mixing device of the mixing apparatus is activated to combine the supplement with a cell culture media in the mixing apparatus and any other media that may be present. At 610, the process ends with an indication that the supplement preparation and sterilization process is complete. In addition, a query may be generated to request input indicative of whether an additional supplement or a temperature sensitive additive is desired.

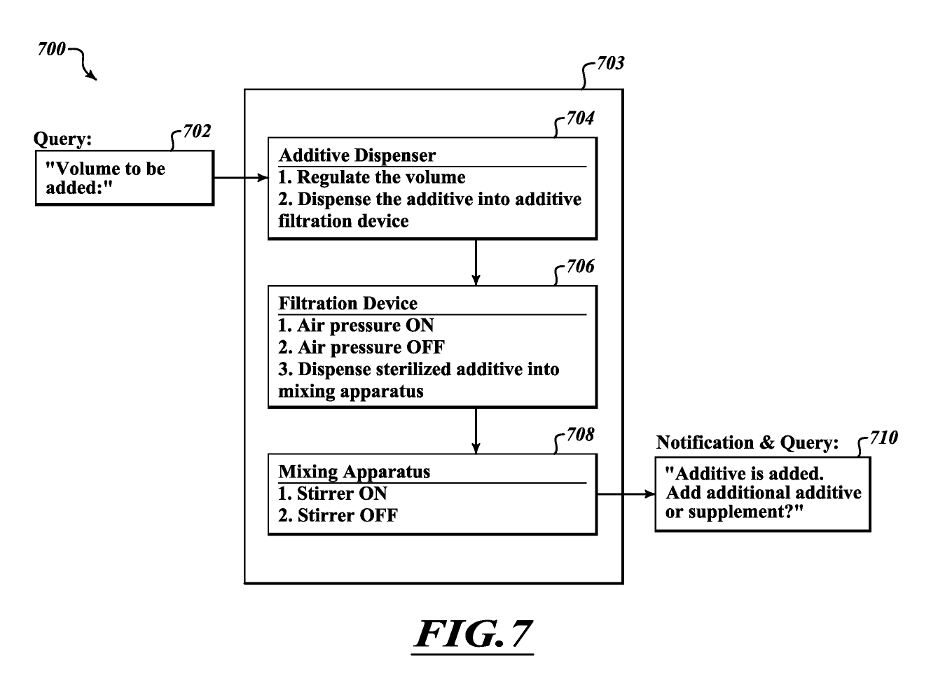

FIG. 7 shows a process flow diagram for preparing a temperature sensitive additive for combination with an agar cell culture media or other cell culture media according to one example embodiment. The process 700 begins at 702 where a query is generated to solicit the amount of the temperature sensitive additive to be processed by a preparation system 703. The query may seek input relating to the weight of the of the temperature sensitive additive, the volume of the temperature sensitive additive (as illustrated in FIG. 7) or other information indicative of the amount of the supplement that is desired to be processed. Upon receiving such input, the corresponding amount of the temperature sensitive additive is apportioned from a supply of the supplement at 704 and discharged into an additive filtration device for further processing.

At 706, an internal chamber of the additive filtration device is pressurized to urge the temperature sensitive additive through at least one filter element to sterilize the temperature sensitive additive. After the temperature sensitive additive is sterilized, the pressurized air source may be deactivated or otherwise isolated from the internal chamber of the additive filtration device. The temperature sensitive additive may be discharged from the additive filtration device toward and ultimately into the mixing apparatus. At 708, a stirrer or mixing device of the mixing apparatus is activated to combine the temperature sensitive additive with a cell culture media in the mixing apparatus and any other media that may be present. At 710, the process ends with an indication that the temperature sensitive additive preparation and sterilization process is complete. In addition, a query may be generated to request input indicative of whether other supplements or additives are desired.

The systems and methods for preparing cell culture dish media described above are particularly well adapted for sterilizing cell culture media, supplements and additives in an efficient and versatile manner to form cell culture dish media having a variety of compositions. The resultant sterilized cell culture dish media is well adapted with respect to uniformity and nutritional content to culture microorganisms in a particularly effective manner. In addition, the systems and methods provide versatile means for preparing cell culture dishes with many different combinations of ingredients without requiring re-sterilization processes that would otherwise be required in more limited systems. The systems described herein include a variety of filtration and sterilization devices for sterilizing different types of media in a parallel relationship to facilitate the preparation of cell culture dishes with different combinations of optional ingredients. In this manner, a user may prepare many different types of cell culture dishes and conduct different kinds of experiments with different parameters all with one convenient cell culture dish media preparation system.

Again, in the above description, certain specific details are set forth in order to provide a thorough understanding of various disclosed embodiments. However, one skilled in the relevant art will recognize that embodiments may be practiced without one or more of these specific details. Moreover, the various embodiments described above can be combined to provide further embodiments. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled.

1. A system for preparing cell culture dish media, the system comprising:
a primary filtration device having at least one membrane filter, the filtration device configured to receive an amount of a cell culture media and sterilize the cell culture media via a heated filtration process;
a dispenser configured to selectively dispense an amount of a desired ingredient for mixing with the cell culture media;
an intermediate receptacle coupled to the primary filtration device and the dispenser to selectively receive the cell culture media and the desired ingredient from the primary filtration device and the dispenser, respectively; and
a mixing chamber positioned downstream of the intermediate receptacle to receive the cell culture media and the desired ingredient and mix the same during a mixing operation to form a cell culture dish media, the mixing chamber including an outlet to selectively discharge the cell culture dish media.

2. The system of claim 1, further comprising:
a second filtration device positioned between the dispenser and the intermediate receptacle to selectively receive the desired ingredient from the dispenser and filter sterilize the desired ingredient prior to introduction into the intermediate receptacle.

3. The system of claim 2 wherein the second filtration device is a supplement filtration device which is configured to receive and filter sterilize a supplement for mixing with the cell culture media.

4. The system of claim 2 wherein the second filtration device is an additive filtration device which is configured to receive and filter sterilize a temperature sensitive additive at or below an ambient temperature for mixing with the cell culture media.

5. The system of claim 2, further comprising:
a third filtration device coupled to the intermediate receptacle, the third filtration device configured to receive an amount of another desired ingredient, sterilize the other desired ingredient via a filtration process and discharge the other desired ingredient into the intermediate receptacle.

6. The system of claim 5 wherein the primary filtration device, the second filtration device and the third filtration device are arranged in a parallel relationship to discharge different media into the intermediate receptacle independently of each other.

7. The system of claim 1, further comprising:
a second dispenser coupled to the intermediate receptacle to receive a non-filterable media and selectively dispense the non-filterable media into the intermediate receptacle for sterilization via pressurized steam.

8. The system of claim 1, further comprising:
a pressurized steam source coupled to the intermediate receptacle to selectively sterilize the intermediate receptacle with pressurized steam during a sterilization operation.

9. The system of claim 1, further comprising:
a second heat source coupled to the intermediate receptacle to selectively maintain the cell culture media at an elevated temperature of at least 45° C. when the cell culture media is received in the intermediate receptacle.

10. The system of claim 1, further comprising:
a mixing apparatus upstream of the primary filtration device to prepare the cell culture media for introduction into the primary filtration device, the mixing apparatus including a cell culture media powder dispenser and a distilled water dispenser for selectively dispensing amounts of cell culture media powder and distilled water into a mixing chamber of the mixing apparatus.

11. The system of claim 10 wherein the mixing apparatus includes a heating element configured to maintain the cell culture media at an elevated temperature of at least 45° C. while discharging the cell culture media from the mixing chamber to the primary filtration device.

12. The system of claim 1, further comprising:
a pressurized air source coupled to at least the primary filtration device to selectively urge cell culture media through the at least one membrane filter during a filtration operation.

13. The system of claim 12 wherein the pressurized air source is configured to subject the cell culture media at least temporarily to a pressure between about 0.1 MPa and about 0.7 MPa to urge the cell culture media through the at least one membrane filter.

14. The system of claim 1 wherein the primary filtration device includes an upstream membrane filter to filter particles of a size greater than a first threshold size and a downstream membrane filter to filter particles of a size greater than a second threshold size, the first threshold size of the upstream membrane filter being between about 0.35 μm and about 1.20 μm and the second threshold size of the downstream membrane filter being between about 0.10 μm and about 0.40 μm.

15. The system of claim 1, further comprising:
a plurality of valves, at least one valve coupled between the intermediate receptacle and each of the primary filtration device and the mixing chamber; and
a control system coupled to the plurality of valves to selectively control the amount of the cell culture media introduced into the intermediate receptacle and discharged into the mixing chamber.

16. The system of claim 15 wherein the control system is further coupled to an air release valve to selectively release pressurized air into the primary filtration device to urge cell culture media through the at least one membrane filter during a filtration operation.

17. The system of claim 15 wherein the control system is further coupled to a steam release valve to selectively release pressurized steam into the intermediate receptacle.

18. The system of claim 15 wherein the control system is further coupled to the heat source of the primary filtration device to regulate a temperature of the cell culture media as the cell culture media passes through the at least one membrane filter during a filtration operation.

19. The system of claim 18 wherein the heat source is configured to maintain the temperature of the cell culture media at or above about 45° C. as the cell culture media passes through the at least one membrane filter during the filtration operation.

20. The system of claim 15 wherein the control system is further coupled to a discharge valve at the outlet of the mixing chamber to selectively discharge the cell culture dish media.

21. The system of claim 15 wherein the control system is further coupled to a conveyance system to selectively position a series of cell culture dishes intermittingly below the outlet of the mixing chamber to selectively receive the cell culture dish media to form cell culture dishes.

22. The system of claim 1 wherein the intermediate receptacle includes a tubular structure that slopes downwardly toward the mixing chamber.

23. A method of preparing cell culture dish media, the method comprising:
sterilizing cell culture media through at least one membrane filter of a primary filtration device in a heated filtration process;
discharging the sterilized cell culture media into a mixing chamber downstream of the primary filtration device;
mixing the sterilized cell culture media with at least a first ingredient in the mixing chamber to form a cell culture dish media; and
selectively discharging the cell culture dish media from the mixing chamber to form one or more cell culture dishes.

24. The method of preparing cell culture dish media of claim 23, further comprising:
sterilizing the first ingredient through a second filtration device prior to introduction in the mixing chamber.

25. The method of preparing cell culture dish media of claim 24 wherein sterilizing the first ingredient through the second filtration device prior to introduction in the mixing chamber includes sterilizing a supplement through at least one membrane filter of an supplement filtration device.

26. The method of preparing cell culture dish media of claim 24 wherein sterilizing the first ingredient through the second filtration device prior to introduction in the mixing chamber includes sterilizing a temperature sensitive additive at or below an ambient temperature through at least one membrane filter of an additive filtration device.

27. The method of preparing cell culture dish media of claim 24, further comprising:
mixing the sterilized cell culture media with at least a second ingredient in the mixing chamber to form a cell culture dish media; and
sterilizing the second ingredient through a third filtration device prior to introduction in the mixing chamber.

28. The method of preparing cell culture dish media of claim 23, further comprising:
discharging the sterilized cell culture media into an intermediate receptacle prior to introduction into the mixing chamber; and
sterilizing the intermediate receptacle with pressurized steam after the sterilized cell culture media is discharged into the mixing chamber.

29. The method of preparing cell culture dish media of claim 23, further comprising:
dispensing a non-filterable cell culture media into an intermediate receptacle; and
introducing pressurized steam into the intermediate receptacle to sterilize the non-filterable cell culture media.

30. The method of preparing cell culture dish media of claim 23, further comprising:
maintaining the cell culture media at an elevated temperature of at least about 45° C. while discharging the cell culture media into the primary filtration device.

31. The method of preparing cell culture dish media of claim 23, further comprising:
maintaining the cell culture media at an elevated temperature of at least about 45° C. while sterilizing the cell culture media through the at least one membrane filter of the primary filtration device.

32. The method of preparing cell culture dish media of claim 31, further comprising:
maintaining the sterilized cell culture media at an elevated temperature of at least about 45° C. while the cell culture media is received in an intermediate receptacle prior to introduction into the mixing chamber.

33. The method of preparing cell culture dish media of claim 32, further comprising:
maintaining the sterilized cell culture media at an elevated temperature of at least about 45° C. while the cell culture media is mixed in the mixing chamber.

34. The method of preparing cell culture dish media of claim 23, further comprising:
dispensing amounts of cell culture media powder and distilled water into a mixing chamber of an upstream mixing apparatus; and
mixing the determined amounts of cell culture media powder and distilled water to prepare the cell culture media for introduction into the primary filtration device.

35. The method of preparing cell culture dish media of claim 23, further comprising:
urging the cell culture media through the at least one membrane filter of the primary filtration device with pressurized air.

36. The method of preparing cell culture dish media of claim 23, further comprising:
conveying a series of dishes intermittingly below an outlet of the mixing chamber to selectively receive the cell culture dish media to form cell culture dishes.

37. The method of preparing cell culture dish media of claim 23 wherein sterilizing the cell culture media, discharging the sterilized cell culture media into the mixing chamber, mixing the sterilized cell culture media with at least the first ingredient in the mixing chamber to form a cell culture dish media and selectively discharging the cell culture dish media from the mixing chamber to form one or more cell culture dishes are automated to produce one or more cell culture dishes each having a determined volume of the cell culture dish media in response to a preparation instruction.

38. A cell culture dish prepared by the method of claim 23.
